(19) United States
(12) Patent Application Publication
(10) Pub. No.: US 2009/0117350 A1
(43) Pub. Date: May 7, 2009

(54) Aqueous ink composition, method for detecting ink using same, ink jet recording apparatus, and recording

(75) Inventors: Takashi Oyanagi, Matsumoto-shi (JP); Kiyohiko Takemoto, Matsumoto-shi (JP); Chiyoshige Nakazawa, Matsumoto-shi (JP)

Correspondence Address: EDWARDS ANGELL PALMER & DODGE LLP, P.O. BOX 55874, BOSTON, MA, 02205, US

(73) Assignee: Seiko Epson Corporation, Tokyo (JP)

(21) Appl. No.: 12/291,374

(22) Filed: Nov. 7, 2008

(30) Foreign Application Priority Data
Nov. 7, 2007 (JP) 2007-289555

(51) Int. Cl. B32B 3/10; G01N 27/327; C12Q 1/00; C12Q 1/48; G01D 11/00; C09D 11/00; C12Q 1/26; C12Q 1/28

(52) U.S. Cl. 428/195.1; 205/777.5; 435/4; 435/15; 435/25; 435/28; 106/31.13; 347/100

(57) ABSTRACT

A method for detecting an aqueous ink composition contained in an ink container, the aqueous ink composition containing at least one reducing sugar, includes measuring the type or concentration of the reducing sugar contained in the aqueous ink composition, and determining the type or remaining amount of the aqueous ink composition on the basis of the result.

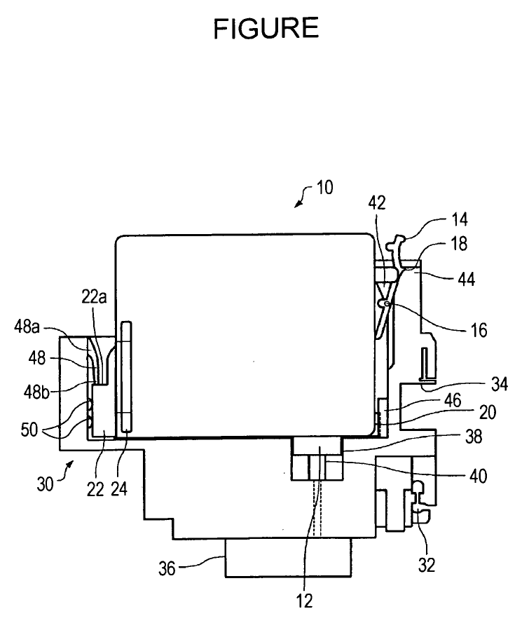

### BACKGROUND

1. Technical Field

The present invention relates to an aqueous ink composition, a method for detecting ink using the aqueous ink composition, an ink jet recording apparatus, and a recording.

2. Related Art

Hitherto, optical detecting methods have been employed for detecting the amounts of ink compositions remaining in cartridges (for example, see Japanese Unexamined Patent Application Publication No. 2000-190523). In the case of using aqueous pigment ink, however, it has been difficult to perform optical measurement because of high absorbance of a colorant unless the ink has been diluted in a solvent in advance.

Ink jet recording apparatuses in the related art have not included units configured to immediately detect misoperation and alert users when ink cartridges have been placed at incorrect positions. Such misoperation has caused failure of ink jet recording apparatuses, leading to loss of opportunity for users. In models using inks of similar colors, such as black, gray, and light black; light cyan, cyan, and blue; or light magenta, magenta, red, and orange, in particular, it has been difficult to distinguish among ink colors on the basis of external appearance, in some cases. Thus, ink cartridges have been placed at incorrect positions, in some cases.

### SUMMARY

An advantage of some aspects of the invention is that it provides an aqueous ink composition and a method for detecting ink using the aqueous ink composition with high accuracy. Furthermore, an advantage of some aspects of the invention is that it provides an ink jet recording apparatus that can immediately detect misoperation and alert users when ink cartridges are placed at incorrect positions.

According to an embodiment of the invention, a method for detecting an aqueous ink composition contained in an ink container, the aqueous ink composition containing at least one reducing sugar, includes measuring the type or concentration of the reducing sugar contained in the aqueous ink composition, and determining the type or remaining amount of the aqueous ink composition on the basis of the result.

It is preferable that the concentration of the sugar contained in the aqueous ink composition be in the range of 0.01% to 1% by weight.

It is preferable that the aqueous ink composition further contain at least one selected from antiseptic agents, bactericides, antimicrobial agents, and antifungal agents.

It is preferable that the measuring the type or concentration of the reducing sugar be performed by an enzyme electrode method or a colorimetric enzymatic method.

It is preferable that the enzyme electrode method be a glucose oxidase method or a glucose dehydrogenase method.

It is preferable that the colorimetric enzymatic method be a hexokinase method or a glucose oxidase/peroxidase method.

According to another embodiment of the invention, an ink jet recording apparatus includes a plurality of ink cartridges. Each of the ink cartridges contains the aqueous ink composition described above. The types or concentrations of the reducing sugars are different depending on the types of the aqueous ink compositions.

It is preferable that the ink jet recording apparatus further include a sensor arranged in each of the ink cartridges so as to be in contact with the aqueous ink composition, each of the sensors being configured to measure the concentration of the sugar.

According to another embodiment of the invention, a recording is formed on a recording medium with the ink jet recording apparatus described above.

### BRIEF DESCRIPTION OF THE DRAWING

The invention will be described with reference to the accompanying drawings, wherein like numbers reference like elements.

FIGURE is a cross-sectional view of a carriage and an ink cartridge mounted on an ink jet recording apparatus according to an embodiment of the invention.

### DESCRIPTION OF EXEMPLARY EMBODIMENTS

Preferred embodiments of the invention will be described in detail below with reference to attached drawings.

First, an ink composition according to this embodiment will be described in detail below.

### 1. Aqueous Ink Composition

An aqueous ink composition according to this embodiment is an ink composition used for ink jet recording and contains at least one reducing sugar.

### 1.1 Reducing Sugar

In the case where a reducing sugar is added to an aqueous ink composition, the sugar concentration in the ink composition is measured by a measurement method, e.g., an enzyme electrode method or a colorimetric enzymatic method, using a redox reaction to determine whether any ink remains or not.

Examples of the reducing sugar include aldoses and ketoses, which are monosaccharides. Examples of aldoses include glyceraldehyde, erythrose, threose, arabinose, xylose, ribose, lyxose, glucose, galactose, mannose, allose, altrose, idose, talose, and gulose. Examples of ketoses include dihydroxyacetone, erythrulose, xylulose, ribulose, fructose, psicose, sorbose, and tagatose.

Among these reducing monosaccharides, the addition of a rare sugar, such as L-glucose, L-mannose, D-psicose, or L-tagatose, which occurs rarely in nature, to the ink permits the distinction between genuine ink and other non-genuine inks. This identification method is based on the fact that there is no natural contamination of the ink with the rare sugar.

Examples of the reducing sugar further include lactose and cellobiose, which are disaccharides.

The concentration of the reducing sugar is preferably in the range of 0.01% to 1% by weight and more preferably 0.02% to 0.6% by weight. A concentration of the reducing sugar exceeding 1% by weight may cause microbial contamination of the ink, thereby degrading the ink. Thus, an antiseptic agent, a bactericide, an antimicrobial agent, or an antifungal agent needs to be added to the ink. At a concentration of the reducing sugar of less than 0.01% by weight, in some cases, the sugar cannot be detected because of an excessively low sugar concentration. Those reducing sugars may be used alone or in combination.

### 1.2 Pigment

The ink composition according to this embodiment may contain a known pigment that can be used in common aqueous ink compositions.

Examples of a pigment used in black ink include C.I. Pigment Blacks 1 (aniline black), 7 (carbon black), and 11 (iron oxide); and furnace black, lamp black, acetylene black, channel black, copper oxide, and titanium oxide.

Examples of a pigment used in yellow ink include C.I. Pigment Yellows 1, 3, 12, 13, 14, 17, 24, 34, 35, 37, 42, 53, 55, 74, 81, 83, 93, 95, 97, 98, 100, 101, 104, 108, 109, 110, 114, 117, 120, 128, 129, 138, 151, 153, 154, 155, and 180.

Examples of a pigment used in magenta ink include C.I. Pigment Reds 1, 2, 3, 5, 7, 12, 17, 22, 23, 31, 38, 48:2 (Ca), 48:4 (Mn), 49:1, 52:2, 53:1, 57:1, 60:1, 63:1, 63:2, 64:1, 81, 83, 88, 101, 104, 105, 106, 108, 112, 114, 122, 123, 146, 149, 166, 168, 170, 172, 177, 178, 179, 184, 185, 190, 193, 202, 209, 219, and 264; and C.I. Pigment Violets 19 and 42.

Examples of a pigment used in blue ink include C.I. Pigment Blues 1, 2, 3, 15, 15:1, 15:2, 15:3, 15:4, 15:6, 16, 17:1, 22, 56, 60, and 63; and C.I. Vat Blues 4 and 60.

Examples of a pigment used in green ink include C.I. Pigment Greens 1, 4, 7, 8, 10, 17, 18, and 36.

The aqueous ink composition according to this embodiment is preferably prepared by, for example, preparing a pigment dispersion containing the foregoing pigment dispersed in an aqueous medium with a dispersant or a pigment dispersion containing a self-dispersible pigment dispersed in an aqueous medium and gradually adding an ink solvent and the like to the pigment dispersion.

### 1.3 Moisturizing Agent

The aqueous ink composition according to this embodiment may contain a moisturizing agent. The addition of the moisturizing agent can prevent the ink from drying, in particular, can prevent the evaporation of water in a nozzle located at an end of an ink-ejecting head to block the clogging of the nozzle.

Examples of the moisturizing agent include water-soluble glycols, such as glycerol, ethylene glycol, diethylene glycol, triethylene glycol, tetraethylene glycol, polyethylene glycol, propylene glycol, dipropylene glycol, tripropylene glycol, 1,3-propanediol, 1,4-butanediol, 1,5-pentanediol, 2,3-butanediol, 2-methyl-2,4-pentanediol, 1,2,6-hexanetriol, pentaerythritol, and meso-erythritol.

The aqueous ink composition according to this embodiment may contain an auxiliary moisturizing agent in order to assist the function of the moisturizing agent.

Examples of the auxiliary moisturizing agent include glucose, mannose, fructose, ribose, xylose, arabinose, galactose, aldonic acid, glucitol, sorbitol, maltose, maltitol, cellobiose, lactose, sucrose, trehalose, and maltotriose. Among these agents, sorbitol and maltitol, which are sugar alcohol, are particularly preferred.

The amount of the moisturizing agent and the auxiliary moisturizing agent added is preferably in the range of 1% to 40% by weight and more preferably 1% to 30% by weight with respect to the total amount of the aqueous ink composition. The moisturizing agent and the auxiliary moisturizing agent may be used alone or in combination.

### 1.4 Penetrating Organic Solvent

The aqueous ink composition according to this embodiment may contain acetylene glycol and an ether derivative thereof dissolved in a penetrating organic solvent. The penetrating organic solvent is preferably at least one selected from glycol monoether derivatives of polyhydric alcohols, 1,2-alkyldiols, and derivatives having a 1,3-propanediol skeleton.

The glycol monoether derivatives of polyhydric alcohols are preferably polyhydric alcohol derivatives each having an alkyl chain with three or more carbon atoms. Examples thereof include ethylene glycol monobutyl ether, diethylene glycol mono-n-propyl ether, ethylene glycol mono-iso-propyl ether, diethylene glycol mono-iso-propyl ether, ethylene glycol mono-n-butyl ether, ethylene glycol mono-tert-butyl ether, diethylene glycol mono-n-butyl ether, triethylene glycol mono-n-butyl ether, diethylene glycol mono-tert-butyl ether, 1-methyl-1-methoxybutanol, propylene glycol mono-tert-butyl ether, propylene glycol mono-n-propyl ether, propylene glycol mono-iso-propyl ether, propylene glycol mono-n-butyl ether, dipropylene glycol mono-n-butyl ether, dipropylene glycol mono-n-propyl ether, and dipropylene glycol mono-iso-propyl ether. The amount of the glycol monoether derivative of polyhydric alcohol added is preferably in the range of 0.5% to 15% by weight with respect to the total amount of the aqueous pigment ink.

As the 1,2-alkyldiols, 1,2-alkyldiols each having 4 to 8 carbon atoms are preferred. Examples thereof include butanediol, pentanediol, hexanediol, heptanediol, and octanediol. Among these, 1,2-hexanediol, 1,2-heptanediol, and 1,2-octanediol each having 6 to 8 carbon atoms are particularly preferred because of high penetrability to recording media. The amount of 1,2-alkyldiol added is preferably in the range of 0.25% to 15% by weight with respect to the total amount of the aqueous pigment ink.

### 1.5. Polymeric Dispersant

The aqueous ink composition according to this embodiment may contain a polymeric dispersant. The addition of the polymeric dispersant permits the foregoing pigment to be uniformly dispersed in an aqueous medium.

Examples of the polymeric dispersant include naturally-occurring polymers and synthetic polymers.

Examples of naturally-occurring polymers include proteins, such as glue, gelatin, casein, and albumin; natural rubber, such as gum arabic and gum traganth; glucosides such as saponin; alginic acid derivatives, such as alginic acid, propylene glycol alginate, triethanolamine alginate, and ammonium alginate; and cellulose derivatives, such as methylcellulose, carboxymethyl cellulose, hydroxyethyl cellulose, hydroxypropyl cellulose, and ethylhydroxy cellulose.

Examples of synthetic polymers include polyvinyl alcohol, polyvinylpyrrolidone, polyacrylic acid, acrylic acid-acrylonitrile copolymers, potassium acrylate-acrylonitrile copolymers, vinyl acetate-acrylic ester copolymers, acrylic acid-acrylic ester copolymers, styrene-acrylic acid copolymers, styrene-methacrylic acid copolymers, styrene-methacrylic acid-acrylic ester copolymers, styrene-α-methylstyrene-acrylic acid copolymers, styrene-α-methylstyrene-acrylic acid-acrylic ester copolymers, styrene-maleic acid copolymers, styrene-maleic anhydride copolymers, vinylnaphthalene-acrylic acid copolymers, vinylnaphthalene-maleic acid copolymers, vinyl acetate-ethylene copolymers, vinyl acetate-fatty acid vinylethylene copolymers, vinyl acetate-maleic acid ester copolymers, vinyl acetate-crotonic acid copolymers, vinyl acetate-acrylic acid copolymers, and salts thereof. Among these, a copolymer of a hydrophobic group-containing monomer and a hydrophilic group-containing monomer and a polymer of a monomer having hydrophobic and hydrophilic groups in its molecule are preferred. These polymer dispersants may be used alone or in combinations.

In the case where the pigment is dispersed with the polymer dispersant, the pigment and the dispersant can be mechanically mixed by the application of a shearing force with a disperser, such as a roll mill or a sand mill, to allow the dispersant to be adsorbed on surfaces of particles of the pigment.

### 1.6 Surfactant

The aqueous ink composition according to this embodiment may contain a surfactant. The addition of the surfactant can increase the penetration speed of the ink into a recording medium.

Examples of the surfactant include anionic surfactants, such as fatty acid salts and alkyl sulfate salts; nonionic surfactants, such as polyoxyethylene alkyl ether and polyoxyethylene phenyl ether; acetylenic glycol-based nonionic surfactants; cationic surfactants; amphoteric surfactants; silicone-based surfactants; phosphorous surfactants; and boron-based surfactants. Among these, silicone-based surfactants are preferably used. As the silicone-based surfactants, polyester-modified silicones and polyether-modified silicones are more preferably used. Polyether-modified polydimethylsiloxane or polyester-modified polydimethylsiloxane is particularly preferably used. Specific examples thereof include BYK-347, 348, BYK-UV3500, 3510, 3530, and 3570 (manufactured by BYK Japan KK).

### 1.7 Antiseptic Agent

The aqueous ink composition according to this embodiment contains the reducing sugar as described above. The reducing sugar may be used as a nutrient source to cause microbial contamination. Thus, a material, for example, an antiseptic agent, a bactericide, an antimicrobial agent, or an antifungal agent, for preventing the microbial contamination of the ink is preferably added to the aqueous ink composition according to this embodiment. Examples of the material having such an effect include sodium benzoate, sodium pentachlorophenate, sodium 2-pyridinethiol-1-oxide, sodium sorbate, sodium dehydroacetate, and 1,2-benzisothiazolin-3-one.

### 2. Method for Detecting Ink

A method for detecting ink according to this embodiment is a method for detecting an aqueous ink composition contained in an ink cartridge. The aqueous ink composition is an aqueous pigment ink composition containing at least one of the reducing sugars described above. The type or concentration of the sugar is measured, and the type or remaining amount of the aqueous ink composition is determined on the bases of the result.

### 2.1 Method for Detecting Remaining Amount of Ink

In a method for determining the remaining amount of the aqueous ink composition, the ink is determined to be present when the sugar concentration in the aqueous ink composition can be measured. The ink is determined to be absent when the sugar concentration in the aqueous ink composition cannot be measured.

The method for detecting ink according to this embodiment includes measuring the sugar concentration in the ink composition by the enzyme electrode method or the colorimetric enzymatic method to determine whether any ink remains or not. The enzyme electrode method or the colorimetric enzymatic method provide high measurement sensitivity; hence, the sugar concentration can be measured with high accuracy even when the sugar concentration is as low as about 0.01% to 1% by weight.

The method for detecting ink when, for example, glucose, which is a reducing sugar, is added to the aqueous ink composition will be described below.

### 2.1.1 Enzyme Electrode Method

Examples of the enzyme electrode method include a glucose oxidase method and a glucose dehydrogenase method.

The glucose oxidase method will be first described. An enzyme electrode (hereinafter, also referred to as a “sensor”) contains glucose oxidase and potassium ferricyanide. When the sensor is immersed in the ink composition, glucose oxidase reacts specifically with glucose contained in the ink composition to generate gluconic acid and electrons. The electrons convert potassium ferricyanide into potassium ferrocyanide. By applying a certain voltage to potassium ferrocyanide, potassium ferrocyanide is converted into potassium ferricyanide to generate an electric current. The sugar concentration contained in the ink composition can be measured by utilizing the fact that the electric current is proportional to the concentration of glucose contained in the ink composition.

Next, the glucose dehydrogenase method will be described below. A sensor contains glucose dehydrogenase and potassium ferricyanide. When the sensor is immersed in the ink composition, glucose dehydrogenase reacts with glucose contained in the ink composition to generate gluconolactone and electrons. By converting ferricyanide ions contained in the sensor into reduced ferrocyanide ions and then oxidizing the ferrocyanide ions to form ferricyanide ions, an electromotive force is generated at the electrode. The sugar concentration contained in the ink composition can be measured by utilizing the fact that the electromotive force is proportional to the concentration of glucose in the ink composition.

The ink composition containing glucose, which is a reducing sugar, has been described above. The type of sugar is not particularly limited as long as the sugar has reducibility. An enzyme electrode containing an enzyme in response to the type of sugar may be used.

In the case of employing an enzyme electrode method such as the glucose oxidase method or glucose dehydrogenase method, the sensor needs to be placed so as to be in contact with the inside bottom of the ink cartridge. Alternatively, the sensor needs to be attached to a position just above the bottom. In this case, the sugar concentration cannot be measured when the ink runs out. Therefore, the presence or absence of remaining ink can be determined.

### 2.1.2 Colorimetric Enzymatic Method

Examples of the colorimetric enzymatic method include a hexokinase method and a glucose oxidase/peroxidase method.

The hexokinase method will first be described. Glucose is allowed to react with hexokinase to form glucose-6-phosphate. Glucose-6-phosphate is converted into NADH and tetrazolium by the action of glucose-6-phosphate dehydrogenase. A colored formazan product formed by the action of diaphorase is quantitatively analyzed by colorimetry.

Next, the glucose oxidase/peroxidase method will be described below. Glucose is formed into gluconic acid and hydrogen peroxide by the action of glucose oxidase. Hydrogen peroxide is allowed to react with a chromogen in a reagent by the action of peroxidase to form a quinone dye. The resulting quinone dye is quantitatively analyzed by colorimetry.

The ink composition containing glucose, which is a reducing sugar, has been described above. The type of sugar is not particularly limited as long as the sugar has reducibility.

In the case of employing the colorimetric enzymatic method such as the hexokinase method or the glucose oxidase/peroxidase method, the degree of color development should be determined by measuring absorbance with visible light having an appropriate wavelength. In this case, thus, an ink jet recording apparatus is slightly large compared with that employing the enzyme electrode method.

### 2.2 Method for Detecting Type of Ink

A method for detecting the type of aqueous ink composition utilizes the fact that enzymes capable of inducing reactions with which oxidase or dehydrogenase is associated are different depending on the types of sugars involved (substrate specificity of enzyme). Thus, different reducing sugars are added to the different types of aqueous ink compositions. When the type of reducing sugar contained in an aqueous ink composition can be detected, an ink cartridge containing the ink is determined to have been placed at a correct position. In contrast, when the type of reducing sugar contained in the aqueous ink composition cannot be detected, the ink cartridge containing the ink is determined to have been placed at an incorrect position. Details of the method for determining the type of aqueous ink composition will be described in section “3. Ink Jet Recording Apparatus”.

### 2.3 Application Range

The remaining amount of dye ink containing dye as a colorant can be detected also by a known method for optically detecting ink because of the low absorbance of the colorant. Thus, the aqueous ink composition according to this embodiment is preferably an aqueous pigment ink containing a pigment as a colorant. Of course, the method for detecting ink according to this embodiment can also be applied to dye ink containing dye as a colorant.

### 3. Ink Jet Recording Apparatus

An ink jet recording apparatus according to this embodiment includes a plurality of ink cartridges. The ink cartridges contain aqueous ink compositions each containing at least one reducing sugar described above. The types of sugars or the concentrations of the sugar vary depending on the types of aqueous ink compositions.

In the ink jet recording apparatus according to this embodiment, the type of ink composition can be determined when different types of ink compositions have different concentrations of the reducing sugar.

For example, ink cartridges containing inks similar in color, e.g., black ink and gray ink, magenta ink and light magenta ink, and cyan ink and light cyan ink, may be placed at a position different from a correct position. In the case where each type of ink composition corresponds to a corresponding one of the concentrations of the sugar, it is possible to immediately detect the misplacement and to provide an error indication when the ink cartridge is placed at a position different from a correct position. This prevents misplacement of the ink cartridge and failures such as inks of different colors becoming mixed.

Alternatively, in the ink jet recording apparatus according to this embodiment, the type of ink composition can be determined when different types of ink compositions contain different types of reducing sugars.

For example, the case where glucose, which is a monosaccharide, is added to black ink and where galactose, which is a monosaccharide, is added to gray ink will be described below. Sensors arranged inside ink cartridges contain enzyme electrodes containing enzymes capable of inducing reactions of the sugars with oxidase or dehydrogenase. The term “enzymes” used here indicates glucose oxidase for glucose and galactose oxidase for galactose. The concentration of glucose in the black ink and the concentration of galactose in the gray ink can be measured on the basis of the foregoing principles. Thereby, the types of aqueous ink compositions can be determined.

In the embodiment described above, for example, when a cartridge containing non-genuine black ink containing another reducing sugar such as mannose is placed, the concentrations of sugars other than glucose cannot be measured because of the substrate specificity of the enzyme. This permits the distinction between genuine ink and other non-genuine inks.

An ink jet recording apparatus according to this embodiment will be described below with reference to the attached drawing.

FIGURE is a cross-sectional view of a carriage and an ink cartridge according to an embodiment of the invention.

An ink cartridge 10 includes an ink supply port 12, a lever 14, a projection 16, a claw 18, a protrusion 20, and a protruding portion 22 configured to form a circuit-board-fixing face, which are arranged outside the container thereof, and a sensor 24 arranged inside the container. The sensor 24 is located just above the bottom of the ink cartridge 10. In the case where the sensor 24 is arranged at this position, when the remaining amount of ink is substantially zero, the ink is not in contact with the sensor 24. The sensor 24 contains at least an enzyme electrode and a reference electrode. In the case where the ink is in contact with the sensor, an electric signal is transmitted to an ink-detecting unit that detects the remaining amount of ink, not shown, on the basis of the foregoing principles. When an electric signal is not transmitted to the ink-detecting unit, the ink is not in contact with the sensor 24. In this case, the ink-detecting unit sends a command to CPU to provide an empty indication that indicates that the remaining amount of ink is substantially zero. Meanwhile, in the case where the concentration of the sugar detected by the ink-detecting unit does not agree with the concentration of a sugar in ink that should be placed, the ink-detecting unit sends a command to CPU to provide an error indication that indicates misplacement of the ink cartridge.

A carriage 30 serves as a substantially box-shaped cartridge holder such that a plurality of the ink cartridges 10 can be inserted into the carriage 30 from above. The carriage 30 includes a recess 32 and a guide surface 34 arranged on a side face near the bottom thereof, the recess 32 engaging a guide member formed by flattening a frame or the like of the recording apparatus to control the path of travel, and the guide surface 34 sliding along a planar surface of the other guide member.

A fitting portion 38 into which the ink supply port 12 configured to feed a recoding head 36 arranged on the undersurface of the carriage 30 with ink is fitted and a channel-forming member 40 are arranged in an accommodation region of the carriage 30 for each of the ink cartridges.

A first groove 42 engaging the projection 16 and an engaging portion 44 engaging the claw 18 are arranged on a wall facing the lever 14 of the ink cartridge 10. A second groove 46 is formed below the first groove 42. The second groove 46 engages with the guiding protrusion 20 of the ink cartridge 10 just before the completion of mounting to prevent wobble of the ink cartridge 10 in the width direction after the completion of the mounting.

A positioning resilient member 48 having an upper end 48a serving as a supporting point and a lower end 48b in contact with the planar surface 22a of the protruding portion 22 is arranged in an accommodation region on another surface of the carriage 30 for each of the ink cartridges. Resilient contacts 50 in contact with electrodes provided on the protruding portion 22 are arranged below the resilient member 48.

### 4. EXAMPLES

While the invention will be described in detail by the following examples, the invention is not limited to these examples.

### 4.1 Example 1

### 4.1.1 Preparation of Ink Composition

A pigment, a dispersant, and a deionized water were mixed together with glass beads (diameter: 1.7 mm, the weight of the glass beads is 1.5 times the mixture) for 2 hours using a sand mill (manufactured by Yasukawa Seisakusyo). The beads were removed by a separator to prepare a pigment dispersion. A solvent and additives other than a pigment and a dispersant in each composition shown in Table 1 or 2 were mixed to completely dissolve the additives in the solvent, thus providing an ink solvent. The pigment dispersion was gradually added dropwise to the ink solvent under stirring. The mixture was stirred at room temperature for 30 minutes after completion of the addition. The mixture was filtered through a 5-μm membrane filter to prepare individual ink compositions A to L. Tables 1 and 2 show the compositions of the inks.

TABLE 1

| | Example 1 | | | | | |
|---|---|---|---|---|---|---|
| | A | B | C | D | E | F |
| C.I. Pigment | 6.0 | — | — | 1.0 | — | — |
| Red 122 | | | | | | |
| C.I. Pigment | — | 4.0 | — | — | 1.0 | — |
| Blue 15:3 | | | | | | |
| C.I. Pigment | — | — | 5.0 | — | — | — |
| Yellow 74 | | | | | | |
| C.I. Pigment | — | — | — | — | — | 3.0 |
| Black 7 | | | | | | |
| Styrene-acrylic | 2.0 | 1.5 | 2.0 | 1.0 | 1.0 | 1.5 |
| acid copolymer | | | | | | |
| ammonium salt | | | | | | |
| Glycerol | 12.0 | 10.0 | 10.0 | 20.0 | 20.0 | 10.0 |
| HS-500 | 8.0 | 10.0 | 10.0 | 8.0 | 8.0 | 10.0 |
| 1,2-Hexanediol | 5.0 | 5.0 | 5.0 | 5.0 | 5.0 | 5.0 |
| Triethanol- | 1.0 | 1.0 | 1.0 | 1.0 | 1.0 | 1.0 |
| amine | | | | | | |
| BYK-348 | 0.5 | 0.5 | 0.5 | 0.5 | 0.5 | 0.5 |
| Glucose | 0.5 | 0.5 | 0.5 | 0.5 | 0.5 | 0.5 |
| Deionized | balance | balance | balance | balance | balance | balance |
| water | | | | | | |

TABLE 2

| | Comparative Example 1 | | | | | |
|---|---|---|---|---|---|---|
| | G | H | I | J | K | L |
| C.I. Pigment | 6.0 | — | — | 1.0 | — | — |
| Red 122 | | | | | | |
| C.I. Pigment | — | 4.0 | — | — | 1.0 | — |
| Blue 15:3 | | | | | | |
| C.I. Pigment | — | — | 5.0 | — | — | — |
| Yellow 74 | | | | | | |
| C.I. Pigment | — | — | — | — | — | 3.0 |
| Black 7 | | | | | | |
| Styrene-acrylic | 2.0 | 1.5 | 2.0 | 1.0 | 1.0 | 1.5 |
| acid copolymer | | | | | | |
| ammonium salt | | | | | | |
| Glycerol | 12.0 | 10.0 | 10.0 | 20.0 | 20.0 | 10.0 |
| HS-500 | 8.0 | 10.0 | 10.0 | 8.0 | 8.0 | 10.0 |
| 1,2-Hexanediol | 5.0 | 5.0 | 5.0 | 5.0 | 5.0 | 5.0 |
| Triethanol- | 1.0 | 1.0 | 1.0 | 1.0 | 1.0 | 1.0 |
| amine | | | | | | |
| BYK-348 | 0.5 | 0.5 | 0.5 | 0.5 | 0.5 | 0.5 |
| Glucose | — | — | — | — | — | — |
| Deionized | balance | balance | balance | balance | balance | balance |
| water | | | | | | |

### 4.1.2 Evaluation Method

An ink-detecting device (“G checker”, manufactured by Gunze Ltd.) was placed on the inside bottom of an ink cartridge that feeds an ink jet recording head with the ink composition. Whether the presence or absence of the ink composition was determined was evaluated. The ink-detecting device included an enzyme electrode. The sugar concentration was determined by measuring a current generated in the electrode.

The results demonstrated that the sugar concentration in the ink composition was successfully detected in any of the ink compositions described in Example 1.

In contrast, in Comparative Example 1, the ink-detecting device did not respond because the ink compositions did not contain a reducing sugar (glucose). A printing apparatus did not operate and provided an error indication and an empty indication of the ink cartridge.

### 4.2 Example 2

### 4.2.1 Preparation of Ink Composition

A pigment, a dispersant, and a deionized water were mixed together with glass beads (diameter: 1.7 mm, the weight of the glass beads is 1.5 times the mixture) for 2 hours using a sand mill (manufactured by Yasukawa Seisakusyo). The beads were removed by a separator to prepare a pigment dispersion. A solvent and additives other than a pigment and a dispersant in each composition shown in Table 3 were mixed to completely dissolve the additives in the solvent, thus forming an ink solvent. The pigment dispersion was gradually added dropwise to the ink solvent under stirring. The mixture was stirred at room temperature for 30 minutes after completion of the addition. The mixture was filtered through a 5-μm membrane filter to prepare individual ink compositions M to S. The ink compositions M to S have different glucose concentrations in the range of 0.5% to 3.5% by weight. Table 3 shows the compositions of the inks.

TABLE 3

| | Example 2 | | | | | | |
|---|---|---|---|---|---|---|---|
| | M | N | O | P | Q | R | S |
| C.I. Pigment | 6.0 | — | — | 1.0 | — | — | — |
| Red 122 | | | | | | | |
| C.I. Pigment | — | 4.0 | — | — | 1.0 | — | — |
| Blue 15:3 | | | | | | | |
| C.I. Pigment | — | — | 5.0 | — | — | — | — |
| Yellow 74 | | | | | | | |
| C.I. Pigment | — | — | — | — | — | 3.0 | 1.0 |
| Black 7 | | | | | | | |
| Styrene-acrylic | 2.0 | 1.5 | 2.0 | 1.0 | 1.0 | 1.5 | 1.0 |
| acid copolymer | | | | | | | |
| ammonium salt | | | | | | | |
| Glycerol | 12.0 | 10.0 | 10.0 | 18.0 | 18.0 | 10.0 | 10.0 |
| HS-500 | 8.0 | 9.0 | 9.0 | 8.0 | 8.0 | 8.0 | 7.0 |
| 1,2-Hexanediol | 5.0 | 5.0 | 5.0 | 5.0 | 5.0 | 5.0 | 5.0 |
| Triethanolamine | 1.0 | 1.0 | 1.0 | 1.0 | 1.0 | 1.0 | 1.0 |
| BYK-348 | 0.5 | 0.5 | 1.0 | 0.5 | 0.5 | 0.5 | 0.5 |
| Proxel XL2 | 0.3 | 0.3 | 0.3 | 0.3 | 0.3 | 0.3 | 0.3 |
| Glucose | 0.5 | 1.5 | 1.0 | 2.5 | 3.0 | 2.0 | 3.5 |
| Deionized water | balance | balance | balance | balance | balance | balance | balance |

### 4.2.2. Evaluation Method

An ink-detecting device (“G checker”, manufactured by Gunze Ltd.) was placed on the inside bottom of an ink cartridge that feeds an ink jet recording head with the ink composition. Whether the presence or absence of the ink composition was determined was evaluated. The ink-detecting device included an enzyme electrode. The sugar concentration was determined by measuring a current generated in the electrode.

The results demonstrated that the sugar concentration in the ink composition was successfully detected in any of the ink compositions described in Example 1. Furthermore, the differences in glucose concentration among these ink compositions were detected. In the case where ink compositions of similar colors, e.g., magenta and light magenta, cyan and light cyan, or black and gray, were placed at incorrect positions, a printing apparatus provided an error indication because measured glucose concentrations were different from specified values. That is, the ink jet printer was controlled so that it did not operate.

The invention is not limited to the foregoing embodiments. Various changes may be made. For example, the invention includes substantially the same configurations as in the embodiments (e.g., configurations providing the same functions, methods, and results, or configurations providing the same advantages and operations). Furthermore, the invention includes configurations in which inessential elements of the configurations described in the embodiments are changed. Moreover, the invention includes configuration providing the same operations or advantages as in the embodiments. In addition, the invention includes configurations in which the configurations described in the embodiments are combined with known techniques.

The entire disclosure of Japanese Patent Application No. 2007-289555, filed Nov. 7, 2007 is expressly incorporated by reference herein.

What is claimed is:

1. A method for detecting an aqueous ink composition contained in an ink container, the aqueous ink composition containing at least one reducing sugar, comprising:
measuring the type or concentration of the reducing sugar contained in the aqueous ink composition, and
determining the type or remaining amount of the aqueous ink composition on the basis of the result.

2. The method according to claim 1, wherein the concentration of the sugar contained in the aqueous ink composition is in the range of 0.01% to 1% by weight.

3. The method according to claim 1, wherein the aqueous ink composition further contains at least one selected from antiseptic agents, bactericides, antimicrobial agents, and antifungal agents.

4. The method according to claim 1, wherein the measuring the type or concentration of the reducing sugar is performed by an enzyme electrode method or a colorimetric enzymatic method.

5. The method according to claim 4, wherein the enzyme electrode method is a glucose oxidase method or a glucose dehydrogenase method.

6. The method according to claim 4, wherein the colorimetric enzymatic method is a hexokinase method or a glucose oxidase/peroxidase method.

7. An ink jet recording apparatus comprising:
a plurality of ink cartridges,
wherein each of the ink cartridges contains the aqueous ink composition according to claim 1, and
wherein the types or concentrations of the reducing sugars are different depending on the types of the aqueous ink compositions.

8. The ink jet recording apparatus according to claim 7, further comprising:
a sensor arranged in each of the ink cartridges so as to be in contact with the aqueous ink composition, each of the sensors being configured to measure the concentration of the sugar.

9. A recording formed on a recording medium with the ink jet recording apparatus according to claim 7.
